(19) United States
(12) Patent Application Publication
(10) Pub. No.: US 2009/0233970 A1
(43) Pub. Date: Sep. 17, 2009

(54) COMBINED NSAID AND ACID BLOCKER FORMULATION AND METHOD

(76) Inventor: ROBERT P. NICKELL, Torrance, CA (US)

Correspondence Address: STETINA BRUNDA GARRED & BRUCKER, 75 ENTERPRISE, SUITE 250, ALISO VIEJO, CA, 92656, US

(21) Appl. No.: 12/046,327

(22) Filed: Mar. 11, 2008

(51) Int. Cl. A61K 31/435; A61K 31/196; A61K 31/407; A61K 31/192; A61K 31/4164; A61K 31/341; A61P 29/00; A61P 25/00; A61K 31/426; A61K 31/421; A61K 31/40; A61K 31/405

(52) U.S. Cl. 514/338; 514/567; 514/411; 514/570; 514/374; 514/569; 514/423; 514/420; 514/400; 514/370; 514/365; 514/471

(57) ABSTRACT

The present invention is directed to co-administration of a non-steroidal anti-inflammatory agent (NSAID) and acid blocking agent for the treatment of pain and inflammation with reduced gastrointestinal irritation. A pharmaceutical composition suitable for the co-administration contains a therapeutically effective amount of at least one non-steroidal anti-inflammatory agent, and a therapeutically effective amount of at least one acid blocking agent. A ratio of the non-steroidal anti-inflammatory agent to acid blocking agent in the composition is within a range that provides greater pain relief and reduction of inflammation with less gastrointestinal irritation than that obtainable by the administration of the non-steroidal anti-inflammatory agent or acid blocking agent alone. Examples of pharmaceutical compositions for co-administration of the agents are those containing ibuprofen and ranitidine (“ibudine”), as well as naproxen and ranitidine (“naprodine”).

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field

The invention generally relates to pharmaceutical compositions for the treatment of pain and inflammation, and methods of treatment with the pharmaceutical compositions.

2. Related Art

Pain-relief compounds and methods for their use have been developed for the treatment of various different painful conditions, such as conditions involving acute and/or chronic pain. Categories of compounds known to be useful for such treatment include steroidal and non-steroidal anti-inflammatory compounds (NSAIDs), opioids, NMDA antagonists, and other analgesic agents. Non-steroidal anti-inflammatory drugs in particular have been found to be useful in the treatment of pain associated with inflammation, such as rheumatoid arthritis, osteoarthritis, headache and migraine pain, post-operative pain, tissue injury, gout, ileus and other painful inflammatory disorders. The non-steroidal anti-inflammatory drugs are widely-used because they are non-narcotic and typically relatively safe, with certain NSAIDs even being available over-the-counter without a prescription. Examples of popular NSAIDs include aspirin, ibuprofen and naproxen.

However, a problem with the use of NSAIDs is that they have been discovered to cause significant adverse drug reactions in the form of severe gastrointestinal irritation in certain circumstances, such as with very high doses or prolonged administration of the NSAIDs. The gastrointestinal irritation can be serious enough to cause gastric injury, including serious ulcers and gastrointestinal bleeding, even resulting in death. Certain precautions can be taken to reduce the chances of gastric injury, such as by advising patients to take NSAIDs only after consuming a meal and/or drinking water, and by limiting the dose of the NSAID and duration over which it is administered. However, the risk of gastric injury continues to limit the use of NSAID compounds to lower doses and shorter durations of administration than what may otherwise be desired to achieve pain relief. Also, some patients and physicians avoid taking and/or prescribing NSAIDs altogether out of concern for the potential gastrointestinal risks. The limitations of NSAIDs are especially concerning for chronic conditions such as rheumatoid arthritis, which require long-term therapy.

Accordingly, there remains a need for pharmaceutical compositions and methods capable of providing pain relief without causing adverse gastroinestestinal side effects. There is also a need for pharmaceutical compositions and methods including NSAIDs that provide satisfactory pain relief without gastrointestinal irritation. Furthermore, there is a need for compositions and methods that allow for the relatively safe administration of increased doses of NSAIDs and/or increased administration duration to provide desired pain treatment.

### BRIEF SUMMARY OF THE INVENTION

The present invention is directed to co-administration of a non-steroidal anti-inflammatory agent (NSAID) and acid blocking agent for the treatment of pain and inflammation with reduced gastrointestinal irritation. In one version, a pharmaceutical composition suitable for the co-administration contains a therapeutically effective amount of at least one non-steroidal anti-inflammatory agent, and a therapeutically effective amount of at least one acid blocking agent. A ratio of the non-steroidal anti-inflammatory agent to acid blocking agent in the composition is within a range that provides greater pain relief and reduction of inflammation with less gastrointestinal irritation than that obtainable by the administration of the non-steroidal anti-inflammatory agent or acid blocking agent alone.

In one embodiment of the invention, a method for treating at least one of pain and inflammation in a patient in need thereof is provided that results in reduced gastrointestinal irritation. The method involves administering to the patient a therapeutically effective amount of at least one non-steroidal anti-inflammatory (NSAID) agent, and a therapeutically effective amount of at least one acid blocking agent. The ratio of the non-steroidal anti-inflammatory agent to acid blocking agent administered to the patient is maintained in a range that provides greater pain relief and reduction of inflammation with less gastrointestinal irritation than that obtainable by administration of the non-steroidal anti-inflammatory agent or acid blocking agent alone.

In an embodiment of a pharmaceutical composition for co-administration of the NSAID and acid blocking agent, the pharmaceutical composition contains a non-steroidal anti-inflammatory agent (NSAID) that is ibuprofen or a pharmaceutically acceptable salt thereof in an amount of from about 100 mg to about 800 mg, and an acid blocking agent that is ranitidine or a pharmaceutically acceptable salt thereof in an amount of from 25 mg to 150 mg. In yet another embodiment, the pharmaceutical composition contains a NSAID that is naproxen or a pharmaceutically acceptable salt thereof in an amount of from about 100 mg to about 500 mg, and an acid blocking agent that is ranitidine or a pharmaceutically acceptable salt thereof in an amount of from about 25 mg to 150 mg. In these embodiments, a ratio of the non-steroidal anti-inflammatory agent to the acid blocker can be selected to be within a range of from about one part by weight of the non-steroidal anti-inflammatory agent to about one-fifth to about one-half parts by weight of the acid blocker, such that the composition provides greater pain relief and reduction of inflammation with less gastrointestinal irritation than that obtainable by administration of the non-steroidal anti-inflammatory agent or acid blocking agent alone.

The present invention is best understood by reference to the following detailed description.

### DETAILED DESCRIPTION OF THE INVENTION

The detailed description set forth below is intended as a description of the presently preferred embodiments of the invention, and is not intended to represent the only form in which the present invention may be prepared or utilized. The description sets forth the functions and sequences of steps for preparing and using the invention. It is to be understood, however, that the same or equivalent functions may be accomplished by different embodiments and that they are also intended to be encompassed within the scope of the invention.

The expression “pharmaceutically acceptable salt” as used herein is meant to refer to those salts of biological compounds which retain the biological effectiveness and properties of the free compound (i.e. free bases and/or acids), and can include pharmaceutically acceptable acid and/or base addition salts, as well as pharmaceutically acceptable cationic and/or anionic salts. Examples of pharmaceutically acceptable salts include, but are not limited to, for example, acid addition salts, such as hydrochloride salts, alkali metal salts, such as sodium and potassium, alkaline earth salts, ammonium salts, and the like.

It should also be understood that the compounds and/or pharmaceutically acceptable salts thereof as described herein may be provided in their hydrate and/or solvate forms.

The expression “therapeutically effective amount” as used herein is meant to refer to an amount of a compound or composition effective to result in the amelioration of symptoms associated with a condition, or to provide a beneficial therapeutic effect, such for example including, but not limited to, at least partial pain relief, reduction of inflammation, reduction in gastrointestinal irritation, and/or protection of the stomach lining.

The expression “gastrointestinal irritation” as used herein is meant to refer to at least one of dyspeptic symptoms, gastroduodenal ulcers, peptic ulcers, perforation of ulcers, gastropathy, upper and/or lower gastrointestinal hemorrhaging, gastroduodenal damage, ulcer complications, stomach erosions and the like.

The expression “co-administration” as used herein is meant to refer to the administration of at least two compounds within the same time frame, such as substantially simultaneously. The expression can refer to the administration of at least two compounds in the same dosage form, substantially simultaneous administration in separate dosage forms, or sequential administration of the compounds within a timeframe selected such that the therapeutic effects of the compounds temporally overlap.

The term “patient” as used herein is meant to refer to a human or non-human mammal capable of receiving treatment with the compositions and methods taught herein.

The term “synergistic effect” as used herein is meant to refer to a therapeutic or other effect achieved by the co-administration of two or more compounds that exceeds a mere additive effect of the compounds.

It has been surprisingly discovered that the gastrointestinal irritation caused by NSAIDs can be reduced by co-administration of the NSAID with an acid blocking agent, thereby allowing for improved treatment of painful and inflammatory conditions. The break-down of the gastric mucosa and stomach lining has been discovered to be decreased by taking the two compounds together, thereby providing gastric protection and reducing gastric irritation, gastric erosion, and lessening the incidence of gastrointestinal ulcers and bleeding. In particular, it has been discovered that the NSAID and acid blocking agent can be co-administered in a ratio that is within a range that provides greater pain relief and reduction of inflammation with less gastrointestinal irritation than that obtainable by the administration of either of the NSAID or acid blocking agent alone. Thus, adverse side effects normally associated with NSAIDs are decreased by administering the NSAID and acid blocking agent together, resulting in improved treatment of pain and inflammation with the NSAID, and also improved patient compliance.

The NSAID used for co-administration with the acid blocking agent can be selected in relation to the particular condition being treated, and preferably has proven efficacy in the treatment of pain and/or inflammation. Examples of NSAIDs (and their brand-names) suitable for co-administration with the acid blocking agent include, but are not limited to, at least one of diclofenac (Cataflam®, Voltaren®, Voltaren SR®), etodolac (Lodine®, Lodine XL®), ibuprofen (Motrin®), fenoprofen (Nalfon®), indomethacin (Indocin®), ketoprofen (Orudis®, Oruvail®), nabumetome (Relafan®), naproxen (Naprosyn®), oxaprozin (Daypro®), sulindac (Clinoril®) and tolmetin (Tolectin®), as well as pharmaceutically acceptable salts of these compounds. In one version, the NSAID co-administered with the acid blocking agent comprises at least one of ibuprofen and naproxen, which drugs are both systemically and locally acting and have high efficacy in controlling pain in humans and animals. Ibuprofen corresponds to the chemical formula 2-(4-isobutylphenyl)propionic acid, and is described in further detail together with its pharmaceutically acceptable salts in, for example, U.K. Patent No. 971,700, which is herein incorporated by reference in its entirety. Naproxen corresponds to the chemical formula [(+)-(S)-6-Methoxy-α-methyl-2-napthaleneacetic Acid], and is described in further detail together with its pharmaceutically acceptable salts in, for example, GB Patent 1211134, which is herein incorporated by reference in its entirety.

The acid blocking agent co-administered with the NSAID is a compound that is capable of inhibiting excess gastric acid production, thereby reducing erosion of the stomach lining and lessening gastric irritation. In one version, the acid blocking agent comprises a proton pump inhibitor, which is a class of compounds that blocks the H+/K+ATPase system to reduce gastric acid secretion. Suitable proton pump inhibitors (and their brand names) include, but are not limited to, at least one of esomeprazole (Nexium®), lansoprazole (Prevacid®), omeprazole (Prilosec®), pantoprazole (Protonix®), and rabeprazole (Achiphex®), as well as pharmaceutically acceptable salts of these compounds. In another version, the acid blocking agent comprises an H₂ receptor antagonist, which is a class of compounds that blocks the action of histamine, thereby decreasing acid production. Suitable H₂ receptor antagonists (and their brand names) include, but are not limited to, at least one of cimetidine (Tagamet®), famotidine (Pepcid®), Nizatidine (Axid®), and ranitidine (Zantac®), as well as pharmaceutically acceptable salts of these compounds. For example, the acid blocking agent co-administered with the NSAID can be ranitidine, which corresponds to the chemical formula (N-[2-[[[5-(Dimethylamino)methyl]-2-furanyl]methyl]thio]ethyl-N′-methyl-2-nitro-1,1-ethenediamine, hydrochloride). The preparation of ranitidine and its pharmaceutically acceptable salts are described in further detail in, for example, the Merck Index, An Encyclopedia of Chemicals, Drugs and Biologicals, 13^{th} Edition, which is herein incorporated by reference in its entirety.

The relative amounts of the NSAID and acid blocking agent administered to the patient are selected according to criteria such as the condition to be treated, the particular NSAID and acid blocking agent being administered, the extent of gastric protection desired, the chronic or acute nature of the condition, and other similar criteria. Generally speaking, a ratio of the NSAID to the acid blocker is within a range of from about one part by weight of the NSAID to about one-fifth to about one-half parts by weight of the acid blocking agent. While this ratio is calculated with respect to the free compound (non-salt form), it should be understood that the equivalent ratio can also readily be determined for pharmaceutically acceptable salts of the compounds by using a ratio of the molecular weights of the salts, as known by those of ordinary skill in the art. This range has been discovered to provide greater pain relief and reduction of inflammation, with less gastrointestinal irritation, that what would otherwise be obtainable by the administration of either the NSAID or acid blocking agent alone. In other words, the administration of these compounds exhibits synergistic effects that exceed the mere additive contribution of the individual components.

Suitable dosages of the NSAID and acid blocking agent for co-administration are similarly selected according to the painful and/or inflammatory condition to be treated, as well as to provide for the synergistic effects in terms of pain relief, reduction in inflammation and reduced gastrointestinal irritation. Generally, a suitable dosage of the NSAID may range from about 50 mg to about 1000 mg, whereas a suitable dosage of the acid blocking agent for co-administration with the NSAID may range from about 20 mg to about 200 mg. For example, a suitable treatment regimen can comprise co-administering naproxen or a pharmaceutically acceptable salt thereof in a dosage of from 100 mg to about 500 mg, such as from about 200 mg to about 250 mg, with ranitidine or a pharmaceutically acceptable salt thereof in a dosage of from about 25 mg to about 150 mg, such as from about 50 mg to about 150 mg. As another example, a suitable treatment regimen can comprise co-administering ibuprofen or a pharmaceutically acceptable salt thereof in a dosage of from about 100 mg to about 800 mg, such as about 200 mg to about 400 mg, with ranitidine or a pharmaceutically acceptable salt thereof in a dosage of from about 25 mg to about 150 mg, such as from about 50 mg to about 100 mg.

The NSAID and acid blocking agent are co-administered to treat patients suffering from any of a variety of different painful and/or inflammatory conditions, including but not limited to acute as well as chronic pain conditions. For example, the compounds can be co-administered to treat pain associated with inflammation in arthritic conditions, including but not limited to at least one of rheumatoid arthritis, Still's disease, osteoarthritis, other arthritic conditions. The compounds can also be co-administered to treat pain and/or inflammation associated with non-arthritic conditions, including but not limited to at least one of musculo-skeletal injury, soft tissue injury, dental pain, post-operative pain, port partum pain, surgical pain, dysmenorrheal, migraine, tension headache, sinus headache and neuralgia. Patients treatable by co-administration of the compounds include human patients suffering from these and other painful and/or inflammatory conditions. Veterinary patients suffering from painful conditions, such as for example any of dogs, cats, horses, livestock and the like, may also receive the NSAID and acid blocking agent co-administration treatment.

In one version, co-administration of the NSAID and acid blocking agent is achieved by formulating the compounds into a pharmaceutical composition. The pharmaceutical composition comprises a dosage form suitable for any of a number of different means of administration, including but not limited to oral, buccal, topical, transdermal, rectal, intravenous, intraperitoneal and inhalable dosage forms. For example, the dosage forms can comprise solid dosage forms, such as at least one of powders, granules, tablets, capsules (e.g. hard and soft gelatin capsules), caplets, cachets, suppositories and pessaries. The dosage forms can also be provided in liquid form, such as for example as solutions, suspensions, emulsions, syrups, elixirs and even pressurized compositions. Other dosage forms can include transdermal forms, such as transdermal patches, as well as aerosolizable forms suitable for pulmonary administration. Sustained release formulations can also be provided. The dosage forms typically comprise dosage units, such as tablets or caplets, which contain the appropriate dosage of the NSAID and acid blocking agent for administration to the patient. Each unit dosage form can comprise up to about 99% by weight of the combined NSAID and acid blocking agent, such as from about 0.03% to about 99% by weight, and even from about 1 to about 80% by weight.

Examples of solid dosage forms of the pharmaceutical composition are those including a pharmaceutically acceptable carrier, which can also optionally include ingredients such as at least one of a flavoring agent, filler, compression aid, binders, disintegrants and encapsulating materials. Suitable carriers and/or ingredients for solid dosage forms can include, but are not limited to, at least one of calcium phosphate, magnesium stearate, talc, sugars, hydrous lactose, anhydrous lactose, ribose, dextrin, starch, gelatin, cellulose, methyl cellulose, carboxymethyl cellulose, microcrystalline cellulose, starch glycolate, polyvinylpyrrolidine, polymers of methacrylic acid and divinylbenzene, waxes and ion exchange resins, among others. In the formulation of powder solid dosage forms, the carrier and active ingredients are finely divided and mixed together, and used to fill capsules, sachets, and the like. In the formulation of tablet solid dosage forms, the active ingredients are mixed with a carrier having suitable compression properties, and then compressed into a desired tablet shape and size. Spray-drying techniques can also be used to provide granules suitable for incorporation into capsules or compression into tablets.

Examples of liquid forms of the pharmaceutical composition are those comprising liquid carriers, including but not limited to water, organic solvents, pharmaceutically acceptable oils and/or fats, and combinations thereof, in which one or more of the active agents are dissolved or suspended. The liquid forms optionally further comprise other suitable pharmaceutically acceptable additives such as solubilizers, emulsifiers, buffering agents, preservatives, sweeteners, flavoring agents, suspending agents, thickening agents, coloring agents, viscosity regulators, stabilizers, osmoregulators, and the like. Some examples of liquid forms suitable for oral administration include but are not limited to: liquid compositions having water as a carrier and including additives such as cellulose derivatives, including carboxymethyl cellulose solutions; compositions having an alcoholic carrier and including mono and polyhydridic alcohols, such as glycerin and non-toxic glycols; and also liquid forms comprising pharmaceutically acceptable oils as a carrier, such as coconut oil, safflower oil and/or arachis oil.

The dosage form can be provided in a regimen as prescribed by a physician or veterinarian depending upon the needs of the patient. As an example, a suitable regimen may comprise the administration of one dosage unit (e.g. a tablet and/or capsule) two to four times per day according to the severity of the pain and/or inflammation and the responsiveness of the patient to the medication.

While formulation of the NSAID and acid blocking agent has been described with regards to the combination of the compounds into a single formulation, it should also be understood that the compounds could be co-administered in separate preparations, such as a first unit dosage form comprising the NSAID, and a second unit dosage form for co-administration with the first unit dosage form comprising the acid blocking agent. Other methods or modes of co-administration not specifically described herein should also be understood to be encompassed by the instant invention.

### EXAMPLES

Preferred embodiments of pharmaceutical compositions suitable for co-administration of the NSAID with the acid blocking agent are described in more detail in the following examples. It should be understood that these examples are meant for illustrative purposes only, and are in no way intended to limit the scope of the invention thereto.

### Example 1

Tables I-IV illustrate tablet formulations that provide for co-administration of the NSAID ibuprofen with the acid blocking agent ranitidine. The tablets were prepared by mixing batches of the ingredients and compressing into the tablet unit dosage forms.

TABLE I

| Tablet Ingredient | Amount |
|---|---|
| Ibuprofen | 250 mg |
| Ranitidine | 75 mg |
| Avicel ® PH-101^{(a)} | 60.5 mg |
| Lactose hydrous USP | 20 mg |
| Explotab ®^{(b)} | 10 mg |
| Magnesium Stearate USP | 2 mg |

^{(a)}microcrystalline cellulose;
^{(b)}starch, glycolate USP

TABLE II

| Tablet Ingredient | Amount |
|---|---|
| Ibuprofen | 200 mg |
| Ranitidine | 50 mg |
| Avicel ® PH-101^{(a)} | 247 mg |
| Anhydrous Lactose USP | 227 mg |
| Amberlite ® IRP 88^{(b)} | 16 mg |
| Magnesium Stearate USP | 4 mg |

^{(a)}microcrystalline cellulose;
^{(b)}methacrylic acid and divinylbenzene polymer

TABLE III

| Tablet Ingredient | Amount |
|---|---|
| Ibuprofen | 400 mg |
| Ranitidine | 100 mg |
| Avicel ® PH-101^{(a)} | 228 mg |
| Anhydrous Lactose USP | 100 mg |
| Amberlite ® IRP 88^{(b)} | 6 mg |
| Magnesium Stearate USP | 3 mg |

^{(a)}microcrystalline cellulose;
^{(b)}methacrylic acid and divinylbenzene polymer

TABLE IV

| Tablet Ingredient | Amount |
|---|---|
| Ibuprofen | 800 mg |
| Ranitidine | 25 mg |
| Avicel ® PH-101^{(a)} | 60 mg |
| Anhydrous Lactose USP | 100 mg |
| Explotab ®^{(b)} | 10 mg |
| Talc USP | 10 mg |
| Magnesium Stearate USP | 3.5 mg |

^{(a)}microcrystalline cellulose;
^{(b)}starch, glycolate USP

### Example 2

Tables V-VIII illustrate tablet formulations that provide for co-administration of the NSAID naproxen sodium with the acid blocking agent ranitidine. The tablets were prepared by mixing batches of the ingredients and compressing into the tablet unit dosage forms.

TABLE V

| Tablet Ingredient | Amount |
|---|---|
| Naproxen sodium | 250 mg |
| Ranitidine | 75 mg |
| Avicel ® PH-101^{(a)} | 60.5 mg |
| Lactose hydrous USP | 20 mg |
| Explotab ®^{(b)} | 10 mg |
| Magnesium Stearate USP | 2 mg |

^{(a)}microcrystalline cellulose;
^{(b)}starch, glycolate USP

TABLE VI

| Tablet Ingredient | Amount |
|---|---|
| Naproxen sodium | 200 mg |
| Ranitidine | 50 mg |
| Avicel ® PH-101^{(a)} | 247 mg |
| Anhydrous Lactose USP | 227 mg |
| Amberlite ® IRP 88 ®^{(b)} | 16 mg |
| Magnesium Stearate USP | 4 mg |

^{(a)}microcrystalline cellulose;
^{(b)}methacrylic acid and divinylbenzene polymer

TABLE VII

| Tablet Ingredient | Amount |
|---|---|
| Naproxen sodium | 220 mg |
| Ranitidine | 100 mg |
| Avicel ® PH-101^{(a)} | 228 mg |
| Anhydrous Lactose USP | 100 mg |
| Amberlite ® IRP 88 ®^{(b)} | 6 mg |
| Magnesium Stearate USP | 3 mg |

^{(a)}microcrystalline cellulose;
^{(b)}methacrylic acid and divinylbenzene polymer

TABLE VIII

| Tablet Ingredient | Amount |
|---|---|
| Naproxen sodium | 500 mg |
| Ranitidine | 25 mg |
| Avicel ® PH-101^{(a)} | 60 mg |
| Anhydrous Lactose USP | 100 mg |
| Explotab ®^{(b)} | 10 mg |
| Talc USP | 10 mg |
| Magnesium Stearate USP | 3.5 mg |

^{(a)}microcrystalline cellulose;
^{(b)}starch, glycolate USP

### Example 3

Table IX illustrates capsule formulations 1-3 that provide for co-administration of the NSAID ibuprofen with the acid blocking agent ranitidine. The capsules were prepared by mixing batches of the ingredients and filling hard gelatin capsules with unit dosages of the mixture.

TABLE IX

| Ingredient | Formulation 1 | Formulation 2 | Formulation 3 |
|---|---|---|---|
| Ibuprofen | 200 mg | 400 mg | 800 mg |
| Ranitidine | 50 mg | 75 mg | 100 mg |
| Lactose hydrous USP | 61.5 mg | 163 mg | 190 mg |
| Maize starch dried | 20 mg | 20 mg | 20 mg |
| USP | | | |
| Talc USP | 10 mg | 20 mg | 10 mg |
| Magnesium stearate | 1 mg | 1 mg | 1 mg |

### Example 4

Table IX illustrates capsule formulations 4-6 that provide for co-administration of the NSAID that is naproxen sodium with the acid blocking agent that is ranitidine. The capsules were prepared by mixing batches of the ingredients and filling hard gelatin capsules with unit dosages of the mixture.

TABLE IX

| Ingredient | Formulation 4 | Formulation 5 | Formulation 6 |
|---|---|---|---|
| Naproxen sodium | 220 mg | 250 mg | 500 mg |
| Ranitidine | 50 mg | 75 mg | 100 mg |
| Lactose hydrous USP | 61.5 mg | 163 mg | 190 mg |
| Maize starch dried | 20 mg | 20 mg | 20 mg |
| USP | | | |
| Talc USP | 10 mg | 20 mg | 10 mg |
| Magnesium stearate | 1 mg | 1 mg | 1 mg |

### Example 5

In this example, a method of preparing capsule formulations for the co-administration of ibuprofen and ranitidine (“ibudine”) is described. A batch of the formulation is prepared by providing 40 grams of ibuprofen, USP powder, 7.5 grams of ranitidine HCl, USP powder, 17.8 grams of lactose monohydrate spray dried powder and 0.5 grams of riboflavin (vitamin B2), USP powder. The ingredients are combined in a mortar using the principles of geometric dilution, and triturated well to reduce particle size. Once the ingredients have been combined and reduced to the desired particle size, the mixture is poured evenly into 100 separate capsules, resulting in capsule unit dosage forms each having 400 mg of ibuprofen and 75 mg of ranitidine HCl.

### Example 6

In this example, a method of preparing capsule formulations for the co-administration of naproxen and ranitidine (“naprodine”) is described. A batch of the formulation is prepared by providing 25 grams of naproxen powder, 7.5 grams of ranitidine HCl, USP powder, 29 grams of lactose monohydrate spray dried powder, and 0.5 grams of riboflavin (vitamin B2), USP powder. The ingredients are combined in a mortar using the principles of geometric dilution, and triturated well to reduce particle size. Once the ingredients have been combined and reduced to the desired particle size, the mixture is poured evenly into 100 separate capsules, resulting in capsule unit dosage forms each having 250 mg of naproxen and 75 mg of ranitidine HCl.

Additional modifications and improvements of the present invention may also be apparent to those of ordinary skill in the art. Thus, the particular combination of compounds and methods of administration described and illustrated herein is intended to represent only certain embodiments of the present invention, and is not intended to serve as limitations of alternative devices and methods within the spirit and scope of the invention. Along these lines, it should be understood that other combinations of NSAIDs and acid blocking agents other than those specifically described can also be used. Also, a pharmaceutical composition used for co-administration of the agents may take any of a variety of dosage forms that are known or later developed in the art. Also, it should be understood that different dosages and/or ratios of the NSAID and acid blocking agent other than those specified may be used depending on the nature and synergistic potential of each particular NSAID and acid blocking agent being used.

1. A pharmaceutical composition for the treatment of pain and inflammation with reduced gastrointestinal irritation, the composition comprising:
(a) a therapeutically effective amount of at least one non-steroidal anti-inflammatory (NSAID) agent; and
(b) a therapeutically effective amount of at least one acid blocking agent,
wherein a ratio of the non-steroidal anti-inflammatory agent to acid blocking agent in the composition is within a range that provides greater pain relief and reduction of inflammation with less gastrointestinal irritation than that obtainable by the administration of the non-steroidal anti-inflammatory agent or acid blocking agent alone.

2. The pharmaceutical composition of claim 1 wherein the non-steroidal anti-inflammatory agent comprises at least one of diclofenac, etodolac, fenoprofen, ibuprofen, indomethacin, ketoprofen, nabumetome, naproxen, oxaprozin, sulindac, tolmetin, and pharmaceutically acceptable salts thereof.

3. The pharmaceutical composition of claim 1 wherein the acid blocking agent comprises at least one of a proton pump inhibitor and an H₂ receptor antagonist.

4. The pharmaceutical composition of claim 3 wherein the acid blocking agent comprises a proton pump inhibitor that is at least one of esomeprazole, lansoprazole, omeprazole, pantoprazole, rabeprazole, and pharmaceutically acceptable salts thereof.

5. The pharmaceutical composition of claim 3 wherein the acid blocking agent comprises a H₂ receptor antagonist that is at least one of cimetidine, famotidine, nizatidine, ranitidine, and pharmaceutically acceptable salts thereof.

6. The pharmaceutical composition of claim 5 wherein the non-steroidal anti-inflammatory agent comprises ibuprofen or a pharmaceutically acceptable salt thereof, and the H₂ receptor antagonist comprises ranitidine or a pharmaceutically acceptable salt thereof.

7. The pharmaceutical composition of claim 5 wherein the non-steroidal anti-inflammatory agent comprises naproxen or a pharmaceutically acceptable salt thereof, and the H₂ receptor antagonist comprises ranitidine or a pharmaceutically acceptable salt thereof.

8. The pharmaceutical composition of claim 1 comprising the non-steroidal anti-inflammatory agent and acid blocker in relative amounts of from about one part by weight of the non-steroidal anti-inflammatory agent and from about one-fifth to about one-half parts by weight of the acid blocking agent.

9. The pharmaceutical composition of claim 1 comprising from about 100 mg to about 500 mg of naproxen or a pharmaceutically acceptable salt thereof, and from about 25 mg to about 150 mg of ranitidine of a pharmaceutically acceptable salt thereof.

10. The pharmaceutical composition of claim 9 comprising from about 200 mg to about 250 mg of naproxen or a pharmaceutically acceptable salt thereof, and from about 50 mg to about 100 mg of ranitidine of a pharmaceutically acceptable salt thereof.

11. The pharmaceutical composition of claim 1 comprising from about 100 mg to about 800 mg of ibuprofen or a pharmaceutically acceptable salt thereof, and from about 25 mg to about 150 mg of ranitidine or a pharmaceutically acceptable salt thereof.

12. The pharmaceutical composition of claim 11 comprising from about 200 mg to about 400 mg of ibuprofen or a pharmaceutically acceptable salt thereof, and from about 50 mg to about 100 mg of ranitidine or a pharmaceutically acceptable salt thereof.

13. The pharmaceutical composition of claim 1, wherein the composition is in a dosage form that comprises at least one of oral, buccal, topical, transdermal, rectal, intravenous, intraperitoneal and inhalable form.

14. The pharmaceutical composition of claim 13 wherein the dosage form comprises from 0.03% to 99% by weight of the non-steroidal anti-inflammatory agent and acid blocking agent.

15. The pharmaceutical composition of claim 13 wherein the composition is in a solid dosage form, and comprises a pharmaceutically acceptable carrier comprising at least one of calcium phosphate, magnesium stearate, talc, sugars, hydrous lactose, anhydrous lactose, ribose, dextrin, starch, gelatin, cellulose, methyl cellulose, carboxymethyl cellulose, microcrystalline cellulose, starch glycolate, polyvinylpyrrolidine, polymers of methacrylic acid and divinylbenzene, waxes and ion exchange resins.

16. The pharmaceutical composition of claim 15 wherein the solid dosage form comprises at least one of a powder, granules, tablet, capsule, suppository and pessary.

17. A method for treating at least one of pain and inflammation in a patient in need thereof with reduced gastrointestinal irritation, the method comprising:
administering to said patient:
(i) a therapeutically effective amount of at least one non-steroidal anti-inflammatory (NSAID) agent; and
(ii) a therapeutically effective amount of at least one acid blocking agent,
wherein the ratio of the non-steroidal anti-inflammatory agent to acid blocking agent is in a range that provides greater pain relief and reduction of inflammation with less gastrointestinal irritation than that obtainable by administration of the non-steroidal anti-inflammatory agent or acid blocking agent alone.

18. The method of claim 17, wherein the patient is suffering from at least one of rheumatoid arthritis, Still's disease, osteoarthritis, other arthritic conditions, pain associated with musculo-skeletal injury, soft tissue injury, dental pain, post-operative pain, port partum pain, surgical pain, dysmenorrheal, migraine, tension headache, sinus headache and neuralgia.

19. The method of claim 17, wherein the pharmaceutical composition comprises a non-steroidal anti-inflammatory agent that is at least one of naproxen and ibuprofen and pharmaceutically acceptable salts thereof, an acid blocking agent that is ranitidine or a pharmaceutically acceptable salt thereof.

20. A pharmaceutical composition for the treatment of pain and inflammation with reduced gastrointestinal irritation, the composition comprising:
(a) a non-steroidal anti-inflammatory agent (NSAID) comprising either (i) ibuprofen or a pharmaceutically acceptable salt thereof in an amount of from about 100 mg to about 800 mg, or (ii) naproxen or a pharmaceutically acceptable salt thereof in an amount of from about 100 mg to about 500 mg; and
(b) an acid blocking agent comprising ranitidine or a pharmaceutically acceptable salt thereof in an amount of from about 25 mg to 150 mg,
wherein a ratio of the non-steroidal anti-inflammatory agent to the acid blocker is within a range of from about one part by weight of the non-steroidal anti-inflammatory agent to about one-fifth to about one-half parts by weight of the acid blocker, and wherein the composition provides greater pain relief and reduction of inflammation with less gastrointestinal irritation than that obtainable by administration of the non-steroidal anti-inflammatory agent or acid blocking agent alone.
